(19) United States
(12) Patent Application Publication
(10) Pub. No.: US 2023/0393119 A1
(43) Pub. Date: Dec. 7, 2023

(54) Portable Closed System for Culturing Cells Using Small Volumes of Capillary Blood

(71) Applicant: Northwestern University, Evanston, IL (US)

(72) Inventor: Thomas McDade, Evanston, IL (US)

(21) Appl. No.: 18/328,967

(22) Filed: Jun. 5, 2023

Related U.S. Application Data
(60) Provisional application No. 63/348,624, filed on Jun. 3, 2022.

(51) Int. Cl. G01N 33/50; B01L 3/00

(52) U.S. Cl. CPC G01N 33/5047; G01N 33/502; B01L 3/502715; B01L 2300/0654; B01L 2200/16; B01L 2200/18; B01L 2300/0864

(57) ABSTRACT

A portable, closed cell incubating cassette system and method including: an inlet to receive a volume of a liquid sample and fluidly connected to one or more incubating channels, each of which including one or more gates located in the one or more incubating channels, each of the gates having an open position and a closed position, wherein each of the gates when in the open position is configured to permit the sample to fluidly connect to a remaining portion of each of the incubating channels, and wherein each of the gates when in the closed position is configured to block the sample from fluidly connecting to the remaining portion of each of the incubating channels; and one or more sample incubation chambers each disposed in each of the incubating channels between the inlet and each of the gates.

### CROSS-REFERENCE TO RELATED APPLICATIONS

This application claims the benefit of U.S. Provisional Application 63/348,624, filed Jun. 3, 2022, which is incorporated herein by reference in its entirety.

### FIELD OF THE INVENTION

The disclosed technology is generally directed to cell culture. More particularly the technology is directed to cell culture of small volumes of blood.

### BACKGROUND

Cell culture systems provide important information on the function of immune cells that is relevant to a wide range of diseases, including risk for infectious diseases, cardiovascular disease, cancer, and adverse birth outcomes. Current methods are labor intensive, require sophisticated laboratory infrastructure, and use large volumes of venous blood. Additional challenges include the cost and burden of blood collection, particularly in populations for whom venipuncture is problematic, such as the obese, elderly, infants, and children.

### BRIEF SUMMARY

The technology disclosed herein addresses these limitations with a portable, closed system for culturing cells that requires only a few drops of blood. Beyond overcoming the limitations of venipuncture, this system has notable implications in assessing vaccine efficacy, immune protection against infectious disease, monitoring inflammation, and forecasting risk for chronic degenerative diseases. The system comprises a liquid-tight cassette outfitted with an inlet for introducing a liquid sample. The inlet is fluidically connected to one or more incubation channels. In one embodiment the one or more incubation channels contain one or more regions, including a sample incubation chamber, wherein the sample is incubated with reagents for a period of time with the assistance of a liquid-tight gate in a closed position. In one embodiment the reagents include ligands and antigens intended to activate cellular processes of interest. In one embodiment the cassette is placed in a portable incubator to control the temperature of the sample during incubation. In another embodiment, the cassette and incubator are configured as a single device. When the gate assumes the open position, the incubation channel is fluidically connected to a sample storage chamber wherein, in one embodiment, the sample may be collected by an absorbent material, dried, and preserved for later analysis. Alternatively, in another embodiment, the cassette is incorporated into a point-of-care instrument for direct analysis.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows a cassette for sample collection, culture, and storage. Panel A shows the front view of the cassette. Panel B shows the side view of the cassette.

FIG. 2 shows a portable incubator for use with the cassette of FIG. 1. Panel A shows the front view with the incubator in the open position. Panel B shows the front view with the incubator closed. Panel C shows the side view with the incubator closed. Exemplary dimensions of the incubator are shown below each view in Panels A-C.

### DETAILED DESCRIPTION

Disclosed herein is a portable, closed cell incubating cassette system. Cell culture systems provide important information on the function of immune cells that is relevant to a wide range of diseases, including risk for infectious diseases, cardiovascular disease, cancer, and adverse birth outcomes. Clinical applications are limited by the requirement for large volumes of venous blood and intensive processing in a laboratory setting. The technology disclosed herein solves this problem in designing a portable, closed system for culturing cells that requires only a few drops of capillary blood that can be easily collected through finger stick or other minimally invasive means. Whole blood enters the system directly with no prior processing. Reaction chambers are pre-loaded with reagents such as antigens or ligands that activate leukocytes during an incubation at controlled temperature. Following incubation, cultured blood is transferred internally to filter paper discs which stop the reaction and subsequently dry and preserve the sample with no additional handling or processing required. Since the sample is dried it is safe and easy to transport to the lab via courier or mail, where it can be analyzed for biomarkers of cellular response to stimulation.

The cassette may include a chemically inert, hydrophobic material that is configured to be liquid-tight except for designated openings and channels. In one embodiment the cassette is made of one or more plastic such as polyethylene, polystyrene, or polypropylene.

The sample, in one preferred embodiment, may include whole blood. In another preferred embodiment, the sample may include capillary blood. In other embodiments, the sample may include venous blood. In other embodiments, the sample may include arterial blood. In yet other embodiments the sample may include plasma or serum. In preferred embodiments the sample may be captured by finger stick or heel stick (e.g., using a lancet or similar device), or other sites of capillary blood collection (e.g., ear, upper arm) using a lancet or microneedle array or Tasso device. In one preferred embodiment the volume of the sample may be about 500 μL. In other embodiments the volume of the sample may be between about 50 μL and about 1000 μL.

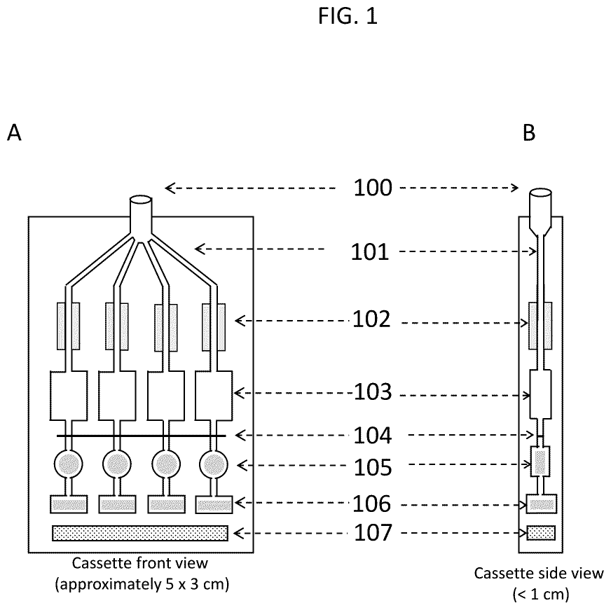

With reference to FIG. 1, there is shown an embodiment of a cassette for blood collections, culture, and storage comprising an inlet (100) wherein a volume of liquid sample may be introduced to the cassette. In one embodiment the inlet feeds into one or more incubating channels (101). In one embodiment, one or more regions of the incubating channel or channels (102) or sample incubation chamber (103) configured to expose the sample to reagents for a period of time with the assistance of one or more gates (104). The gates are configured in the fluidic path of the incubation channel or channels and may assume open or closed positions. In the open position, the gate permits the fluid connection of the sample incubation chamber (103) to the remainder of the channel, including a sample storage chamber (105) and an excess sample chamber (106). In the depicted embodiment, 105 and 106 are in gaseous connection with a desiccant (107). In various embodiments, the cassette may be approximately 3 cm by 5 cm and about 1 cm in thickness, although other dimensions are possible and within the scope of the disclosure.

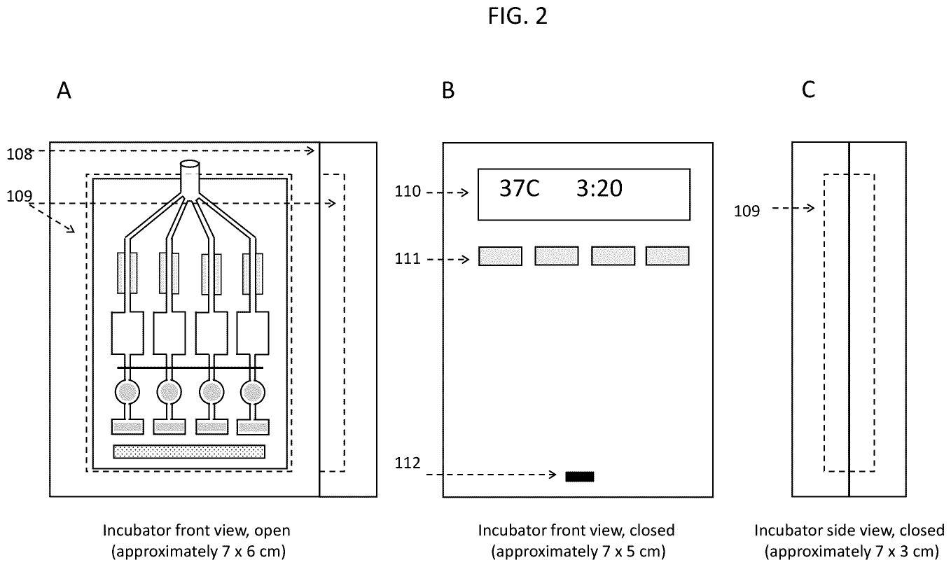

With reference to FIG. 2, there is shown an embodiment of a portable incubator for use with the cassette shown in FIG. 1. In one embodiment, the portable, closed cell culture cassette is a separate component and is configured to be removeable from the incubator. The incubator may include two halves connected to one another with a hinge (108) for opening and closing the incubator. Each half of the incubator may include a recessed area (109) that receives and holds the cassette in place when the incubator is closed. In some embodiments, each half of the incubator may include a heating unit while in other embodiments only one of the halves may include a heating unit. In one embodiment the heating units are powered by rechargeable batteries. In another embodiment, the batteries are recharged by a cable input port (112). In one embodiment, the incubator may include a display for parameters such as the temperature and/or incubation time (110). In one embodiment, the incubator may include buttons (111) to set and control parameters of the incubator such as incubation time and/or temperature. In one embodiment the incubator can heat the cassette to 37° C. In another embodiment, the cassette and incubator are configured to be a single device.

In one embodiment the cell culturing cassette comprises an inlet (100). In some embodiments the sample may be introduced directly into the inlet (100) from a finger prick or indirectly using a pipette or other blood collection device or process documented in the art.

In one embodiment the inlet feeds into one or more incubating channels (101). In some embodiments the liquid sample is drawn through the channel or channels by capillary action. In another embodiment, the liquid sample moves through the channel or channels by applied positive or negative pressure. In one embodiment, positive pressure may be applied through the inlet (100). In another embodiment, negative pressure may be applied through an opening in the cassette body located at or near a terminal end of each of the incubating channels.

In one embodiment, one or more regions of the incubating channel or channels (102) or sample incubation chamber (103) may be configured to contain the entire volume of sample and reagents for a period of time. In some embodiment the reagents may be dried in the incubating channel in advance of introducing a sample. In some embodiments, the dried reagents may be reconstituted with a liquid, including water, buffer solutions, or solvent, by introducing the liquid before the sample is introduced. In other embodiments the reagents may be present as liquids and may be present in, or introduced into, the incubating channel before introduction of the sample. In yet other embodiments, the reagents may be introduced to the incubating channel at the same time as the sample. In certain embodiments, the reagents may be adhered to a wall of a channel of the cassette. In some embodiments, the reagents may include one or more antibodies, ligands, antigens, or growth media. In other embodiments, the reagents may be chosen for positive or negative selection of a specific cells. In some embodiments, those selected cells may be specific leukocyte subsets. In another embodiment, one or more of the incubating channels may be configured with reagents that comprise a positive control or negative control for reagents present in other channels.

The sample may remain in the sample incubation chamber along with reagents for a duration of time sufficient for the cellular process of interest to be activated. In some embodiments this duration of time may range between about 2 hours and about 24 hours, although shorter or longer incubation times are possible and included within the scope of the disclosure.

Ligands are small molecules that may bind receptors and may activate cells. For example, in cell culture different ligands may bind to different white blood cells. In one embodiment, ligands are chosen to activate cells of interest and the results of that cellular activation may be used as indicators of cellular function. In some embodiments, the cellular activation may be indicated by mRNA production, cytokine production, or cell proliferation. In some embodiments, the ligands provide information on the function of T cells, B cell, or cells involved in the regulation of inflammation. In some embodiments, the ligands may include one or more of (or fragments thereof): lipopolysaccharide, hydrocortisone, poly I:C, flagellin, PAM3CSK4, peptidoglycan, zymosan, imiquimod, poly(U)/LyoVec, ODN 2216, anti-CD3/CD28, anti-CD3/CD28/CD137, concanavilin A, phytohemagglutin, wheat germ agglutinin, or pokeweed mitogen.

Antigens are substances that elicit a specific immune response. Antigens include fragments of an infectious agent, including a virus or bacterium, that is recognized by the immune system. The immune system may then react by producing signaling molecules to mobilize cellular and antibody-meditated defenses. In some embodiments, one or more antigens or peptide/antigen pools comprising several fragments of the infectious agent, are used to activate cells and monitor response. The cellular response may provide information on the level of the immune protection against a specific infectious agent. In some embodiments, the cellular response may provide clinically useful information to inform diagnosis of disease as well as level of immune protection following natural infection or vaccination. In some embodiments, the antigens may include one or more of the following (or fragments thereof): SARS-CoV-2, Influenza, Tuberculosis, Herpes simplex virus 1, Herpes simplex virus 2, Epstein-Barr virus, Lyme disease, Human immunodeficiency virus, Rhinovirus, Hepatitis A, Hepatitis B, Tetanus, Diphtheria, Pertussis, Haemophilus Influenzae b, Pneumococcus, Polio, Human papillomavirus, Meningococcal disease, or Measles.

In various embodiments, the cassette may include one or more gates (104) which are configured to be in the fluidic path of the incubation channel or channels and may assume open or closed positions. In the open position, the gate permits the fluid connection of the sample incubation chamber (103) to the remainder of the channel. In the closed position the gate impedes the fluid connection of the sample incubation chamber (103) and the remainder of the channel. In one preferred embodiment, the gate may include a hydrophobic material, such as polyethylene, polystyrene, or polypropylene. In some embodiments the gate is actuated using one or more of manual, electrical or electronic, or magnetic mechanisms. In one preferred embodiment, the gate is arranged in the closed position in the as-shipped or starting configuration of the cassette so that the sample and reagents are contained within the sample incubation chamber (103) during introduction and incubation.

Following a sufficient incubation time for activating a cellular process of interest, the gate is actuated to the open position where the sample incubation chamber (103) has fluidic connection to a sample storage chamber (105). In one embodiment the sample storage chamber is loaded with an absorbent material that can absorb a particular known, fixed amount of the sample (e.g. in some embodiments between 10 μL-250 μL and in one particular embodiment 100 μL). In one embodiment, the absorbent material is a single layer of filter paper (e.g. alpha cellulose-based filter paper such as Whatman #903filter paper or Ahlstrom #226). The absorbent material may be present as a small piece (e.g. a circular disk) that is disposed within the sample storage chamber (105).

The sample storage chamber (105) is fluidically connected to an excess sample chamber (106) that is further loaded with absorbent material (e.g. filter paper as disclosed above) to draw up excess liquid sample.

In the depicted embodiment, elements (105) and (106) are in gaseous connection with a desiccant (107) to facilitate the sample drying on the absorbent material. In some embodiments, the desiccant is one or more of a silica, molecular sieve, and/or clay desiccant.

In one embodiment, the incubated sample can be removed from the cassette for analysis. In those embodiments in which the sample is dried onto absorbent material, the absorbent material with sample dried thereon is removed from the cassette and utilized for further analysis. In some embodiments there is a hinge in the cassette that permits access to the sample storage chamber (e.g. the cassette may include a cover or door that is hingedly attached or the cassette itself may be made as two halves that are separable from one another and which may be hingedly connected to one another). In another embodiment, there is a door over a position of the cassette, e.g. limited to the region of the sample storage chamber, which can be opened to permit access to the dried sample.

In another embodiment, the sample storage chamber (105) is incorporated into a standalone point-of-care analyzer device wherein the incubated sample may be exposed to direct sample analysis for a biomarker of interest following a suitable period of incubation. In one embodiment, the further analysis comprises a lateral flow immunoassay or other miniaturized assay process. One preferred immunoassay is the enzyme-linked immunosorbent spot (ELlspot) assay. In one embodiment, the direct sample analysis is enabled by one or more optically transparent windows for illumination and observation of the sample for optical analysis. Some preferred optical analyses include fluorescence, UV-Vis, Raman, infrared, scattering spectroscopies and microscopies. The optical analysis may be performed in a widefield or confocal configuration in reflectance or transmission geometries.

In some embodiments, the cassette may be provided as part of a kit for obtaining and processing samples. For example, in addition to the cassette, the kit may include one or more of: a sample collection device (e.g. a lancet or Tasso device); containers with reagents; an incubator as disclosed above, including a power source for the incubator (e.g. a battery); instructions for use; and/or software (e.g. a web-based or smartphone app for collecting samples, analyzing results, and/or controlling the cassette and/or incubator).

In some embodiments the cassette system may be used for assessing vaccine efficacy, assessing immune protection against infectious disease, monitoring inflammation during pregnancy, forecasting risk for chronic degenerative diseases, and overcoming limits of venipuncture. In one embodiment, vaccine efficacy may be assessed by measuring the cellular immune response following vaccination against infectious disease threats (e.g., SARS-CoV-2; influenza). For evaluating vaccine effectiveness, it is important to evaluate cellular immunity, as well as serological (i.e., antibody) measures of response. Thus, in some embodiments immune protection against infectious disease may be assessed to determine what is the level of cellular immune protection for one or more individuals following natural infection or vaccination. Information from such assessments may be used to estimate population/herd immunity in the context of epidemics/pandemics of viral agents such as those from the SARS and influenza families. Information regarding the level of protection that can be obtained using the cassette is also important clinical information for individuals who might be at increased risk for adverse outcomes following infection (e.g. for elderly or immunocompromised individuals).

In further embodiments, obtaining information regarding cellular immune protection is important clinically for tailoring vaccine strategy to individual needs. In one embodiment, inflammation during pregnancy may be monitored using the procedures disclosed herein, as dysregulated inflammation can contribute to conditions such as pre-eclampsia, pre-term delivery, and intra-uterine growth restriction, among other adverse birth outcomes. Cell culture systems can provide information on how immune cells respond to pro-inflammatory stimuli, and how effectively they can down-regulate inflammation after it has been turned on. Possessing this information early in pregnancy can be used clinically to reduce the risk of adverse birth outcomes associated with dysregulated inflammation.

In another embodiment, the cassette system may be used for forecasting risk for chronic degenerative diseases. A measure of chronic inflammation, C-reactive protein (CRP), is now widely measured clinically along with blood pressure and lipids to predict risk of cardiovascular and metabolic diseases associated with aging. A functional measure of how an individual regulates inflammation, as is provided by cell culture, provides important diagnostic information on future risk for chronic disease above and beyond CRP. In yet another embodiment, the cassette system may be used to overcome limits of venipuncture. Drawing venous blood for diagnostic and laboratory testing can be difficult with obese individuals and the elderly as well as with infants and children. However, using small volumes of capillary blood as disclosed herein greatly reduces the barriers to implementation for populations such as these.

### Examples

The following are non-limiting descriptions of exemplary procedures for carrying out embodiments of the disclosure:

### Example 1: Method of using the portable, closed cell incubator

Reaction chambers in a small cartridge (approximately 3×5 cm) are pre-loaded with ligands (e.g., lipopolysaccharide) or antigens (e.g., SARS-CoV-2 spike peptide pool). Multiple reaction chambers allow for the simultaneous measurement of responses to different ligands/antigens, as well as positive and negative controls. A small volume (e.g. <500 μL) of whole blood is collected through finger stick, heel stick, or other means, and placed directly into the cassette or transferred by pipet. The cassette is placed inside a small incubator (powered by rechargeable battery or outlet) which maintains a constant temperature of 37° C. Blood flows to the reaction chambers where receptors on leukocytes engage ligand/antigen and activate cells. Incubation times can range from 4 to 24 hours. After incubation, a gate opens and sample flows from the reaction chamber to filter paper (or other absorbent material) that captures a fixed volume of sample. The process of drying the sample stops the reaction and preserves the sample for transport. In the lab, the cassette is opened, filter paper with sample is removed and reconstituted in buffer, and proteins and/or mRNA transcripts are quantified with established protocols as measures of response to stimulation. It may also be possible to integrate the cell culture system into a point-of-care analyzer that directly quantifies response biomarkers without the need for preservation of sample on filter paper.

### Example 2: Cell Culture for Small Volumes of Whole Blood

This example demonstrates a cell culture method that is ideally suited to a portable, closed cell incubator. A “field-friendly” system for measuring the cellular immune response to SARS-CoV-2 in small volumes of whole blood. Whole blood is collected via finger stick or with the Tasso device, stimulated with a pool of SARS antigens, and transferred to filter paper as dried blood spots to stabilize and transport samples with minimal post-culture processing. Cytokine production or gene expression is assessed to characterize the magnitude and pattern of response.

Reagents and Materials
SARS antigen Miltenyi Biotec PepTivator SARS-CoV-2 Prot_S AY.1 WT Reference Pool (130-129-564).
Water Deionized water, endotoxin free.
Normal saline Quality Biological (114-055-721), 9 g/dL NaCl, endotoxin-free
Sodium heparin Sigma-Aldrich (2106-10VL), heparin sodium salt, endotoxin-free, ≥300 USP/vial. Add 500 μL normal saline to vial (≥600 USP/mL). Store at RT. Do not freeze.
Plastics For aliquots of storage stock: Axygen (MCT175C5), 1.7 mL micro-centrifuge tube, sterile, non-pyrogenic
For finger stick protocol: Nalgene (5000-1012), 1.5 mL cryogenic vial, sterile
For whole blood protocol: Corning (430659), 2.0 mL cryogenic vial, non-pyrogenic, sterile
For blood collection with Tasso device:
Capillary tubes PTS Diagnostics (BS002865), 30 uL capillary tubes
Collection device Tasso+
Filter papers Whatman #903 (10534612), protein saver card
Protocol 1: Tasso blood collection
I. Prepare blood collection tube
1) Add 15 μL heparin (>600 USP/mL; final concentration with 500 uL whole blood: >18 USP/mL).
II. Prepare culture tubes
1) Allow lyophilized PepTivator stock to come to room temperature (30 minutes).
Reconstitute stock (6 nmol or 10 ug) with 200 μL sterile water (sH2O). Vortex to dissolve. Final concentration: 30 nmol/mL or −50 ug/mL of each peptide.
2) Make storage stock: 200 μL reconstituted stock+800 μL NaCl (1:5; 10 ug/mL). Aliquot 100 μL into micro-centrifuge tubes. Store at −80° C.
3) Use 1.5 mL Nalgene cryogenic vial for cultures.
4) Dilute storage stock: add 150 μL normal saline to 100 μL storage stock (4 ug/mL; add saline directly to micro-centrifuge tube).
5) Add 50 μL of diluted stock to culture tube (4 ug/mL; 0.2 ug in 50 μL. Mix gently by hand.
III. Prepare control tubes
1) Use 1.5 mL cryogenic vial. Add 50 μL normal saline.
IV. Prepare positive control tubes
V. Prepare cultures
1. Collect 500 μL whole blood in heparinized microtainer using Tasso device.
2. Transfer 100 μL whole blood to control and culture tubes immediately after collection. Cover. Gently swirl. Final dilution: 2 ug/mL relative to whole blood (0.2 ug+100 μL).

3. Incubate 6 hours at 37° C.

4. Label filter paper with sample ID.

Gently swirl culture tube. Set pipet to 65 μL. With forward pipetting, aspirate entire sample (slowly move tip around bottom of tube). Transfer to filter paper. Allow to dry overnight, then store at −20° C.

Reference: McDade, T. W., Aronoff, J. E., Leigh, A. K., Finegood, E. D., Weissman-Tsukamoto, R. M., Brody, G. H., & Miller, G. E. (2021). Out of the laboratory and into the field: Validation of portable cell culture Protocols. Psychosomatic medicine, 83(3), 283-290, which is incorporated by reference herein in its entirety.

### Example 3: Point-of-Care Analyzer with Incorporated Cell Culture Incubator

The closed cell culture cassette will be contained inside of a point-of-care analyzer. A small volume of blood enters the cassette inlet, cells engage ligand/antigen while incubating at 37C, sample is transferred internally to a strip, or another reaction chamber, where a lateral flow immunoassay (or other miniaturized assay process) is used to quantify the response to activation. The enzyme-linked immunosorbent spot (ELIspot) assay is another potential assay to quantify response to cellular activation.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

What is claimed is:

1. A portable, closed cell incubating cassette system, comprising:
an inlet configured to receive a volume of a liquid sample and which is fluidly connected to one or more incubating channels,
each of the one or more incubating channels comprising one or more gates located in the one or more incubating channels, each of the one or more gates having an open position and a closed position,
wherein each of the one or more gates when in the open position is configured to permit the sample to fluidly connect to a remaining portion of each of the one or more incubating channels, and
wherein each of the one or more gates when in the closed position is configured to block the sample from fluidly connecting to the remaining portion of each of the one or more incubating channels; and
one or more sample incubation chambers each disposed in each of the one or more incubating channels between the inlet and each of the one or more the gates.

2. The system of claim 1, further comprising one or more sample storage chambers fluidly connected to each of the one or more sample incubation chambers via each of the one or more gates, wherein each of the one or more sample storage chambers is configured to collect the liquid sample in a fixed, known volume and comprises an absorbent material.

3. The system of claim 2, further comprising one or more excess sample chambers fluidly connected to each of the one or more sample storage chambers.

4. The system of claim 1, further comprising a desiccant having gaseous connection to each of the one or more sample storage chambers and each of the one or more excess sample chambers, wherein the desiccant is configured to absorb moisture from at least one of each of the one or more sample storage chambers or each of the one or more excess sample chambers.

5. The system of claim 1, further comprising a portable incubator configured to warm the cassette to a constant and controlled temperature for a duration of time.

6. The system of claim 5, wherein the portable incubator is powered by a rechargeable battery.

7. The system of claim 5, wherein the cassette system is disposed within the incubator as a separate component.

8. The system of claim 1, wherein each of the one or more channels is preloaded with at least one reagent.

9. The system of claim 8, wherein the at least one reagent includes at least one of an antibody, a ligand, an antigen, or a growth medium.

10. The system of claim 1, wherein the liquid sample moves through the cassette system by at least one of capillary action, positive pressure, or negative pressure.

11. The system of claim 1, wherein the cassette system comprises a door configured to hingedly open to provide access to the cassette system.

12. The system of claim 1, wherein the sample comprises a blood sample.

13. A point-of-care analyzer comprising the system of claim 1, wherein the point-of-care analyzer is configured to detect a particular biomarker in the sample.

14. A method of culturing cells using a portable, closed cell incubating cassette system, comprising:
(i) introducing a sample to an inlet of the cassette system configured to receive a volume of a liquid sample and which is fluidly connected to one or more incubating channels,
each of the one or more incubating channels comprising one or more gates located in the one or more incubating channels, each of the one or more gates having an open position and a closed position,
wherein each of the one or more gates when in the open position is configured to permit the sample to fluidly connect to a remaining portion of each of the one or more incubating channels, and
wherein each of the one or more gates when in the closed position is configured to block the sample from fluidly connecting to the remaining portion of each of the one or more incubating channels; and
one or more sample incubation chambers each disposed in each of the one or more incubating channels between the inlet and each of the one or more the gates;
(ii) incubating the sample in each of the one or more sample incubation chambers for a period of time; and
(iii) collecting a particular amount of the incubated sample in one or more sample storage chambers fluidly connected to the one or more sample incubation chambers.

15. The method of claim 14, further comprising opening the cassette and removing the sample from each of the one or more sample storage chambers.

16. The method of claim 15, wherein collecting a particular amount of incubated sample further comprises:
collecting the particular amount of incubated sample by drying the sample on an absorbent material contained within each of the one or more storage chambers, and
wherein opening the cassette and removing the sample from each of the one or more sample storage chambers further comprises:
removing the absorbent material including the sample from each of the one or more sample storage chambers.

17. The method of claim 14, wherein introducing a sample to an inlet further comprises:
introducing a sample comprising whole blood to the inlet.

18. The method of claim 17, wherein introducing a sample comprising whole blood to the inlet further comprises:
introducing the sample comprising a sample volume of whole blood that is equal to or less than 500 μL to the inlet.

19. The method of claim 14, wherein a portion of each of the one or more incubating channels comprises antibodies, and
wherein introducing a sample to an inlet further comprises:
at least one of capturing specific cells for removal or enriching the sample using the antibodies in each of the one or more incubating channels.

20. The method of claim 14, wherein a portion of each of the one or more incubating channels comprises at least one of a ligand or an antigen, and
wherein incubating the sample in each of the one or more sample incubation chambers further comprises:
incubating the sample in each of the one or more sample incubation chambers in the presence of at least one of the ligand or the antigen to activate one or more cells present in the sample.
